(19) United States
(12) Patent Application Publication
(10) Pub. No.: US 2014/0121653 A1
(43) Pub. Date: May 1, 2014

(54) OPHTHALMIC LASER TREATMENT APPARATUS

(71) Applicant: NIDEK CO., LTD., Gamagori-shi (JP)

(72) Inventors: Hitoshi ABE, Okazaki-shi (JP); Shinichi MATSUURA, Toyokawa-shi (JP)

(21) Appl. No.: 14/063,097

(22) Filed: Oct. 25, 2013

(30) Foreign Application Priority Data
Oct. 31, 2012 (JP) 2012-239698
Oct. 31, 2012 (JP) 2012-239863

(51) Int. Cl. A61F 9/008

(52) U.S. Cl. CPC A61F 9/008
USPC 606/4

(57) ABSTRACT

An ophthalmic laser treatment apparatus for irradiating a laser beam to a patient's eye from a tip of a probe, includes: a probe connector to which fibers of the probe are connected; a splitting unit for splitting a laser beam from a laser source into a plurality of beams; and a control unit for controlling the apparatus. The control unit receives a command to select the number of laser beams to be irradiated from the probe tip; drives the splitting unit according to the received command to switch the number of delivered beams corresponding to the number of fibers to which the laser beam from the laser source will be delivered and increases output of the laser source when the number of delivered beams is switched to an increased number and decreases the output of the laser source when the number of delivered beams is switched to a decreased number.

### CROSS-REFERENCE TO RELATED APPLICATIONS

This application claims priority from Japanese Patent Applications No. 2012-239698 and No. 2012-239863, filed Oct. 31, 2012, the contents of which are hereby incorporated herein by reference in its entirety.

### BACKGROUND

This disclosure relates to an ophthalmic laser treatment apparatus arranged to deliver a laser beam to a fiber of a probe and irradiate the laser beam to a patient's eye through a tip of the probe.

As one of ophthalmic treatment methods, there is known a method for treating a patient's eye by inserting a tip of a probe in a patient's eye, and irradiating a laser beam through the tip of the probe inserted. For instance, an apparatus disclosed in Japanese Translation of PCT International Application Publication No. JP 2000-500043 includes a splitting means (a coupler or a diffraction element), a movement member, and a plurality of fibers. The splitting means splits a laser beam. The movement member moves the splitting means. The plurality of fibers deliver a laser beam. When an operator moves the movement member, an irradiation pattern of a single spot and an irradiation pattern of multiple spots are changed or switched from one to another. In the single-spot irradiation pattern, the laser beam is irradiated to one position. In the multi-spot irradiation pattern, the laser beam is irradiated to a plurality of positions.

### SUMMARY

One aspect in the aforementioned conventional apparatus is mentioned below. When the irradiation patterns of laser beams are switched over by driving the splitting means, the energy of each laser beam to be irradiated from the probe varies. For instance, if a laser beam generated in a laser source is split into N pieces of laser beams by the splitting means, the energy of each laser beam is 1/N as compared with the case where a laser beam is not split. Accordingly, when the operator changes from an irradiation pattern to another, treatment effects available from each laser beam vary. Specifically, it is difficult for the conventional art to switch between the irradiation patterns of laser beams to be irradiated from the tip of the probe while restraining the variation in treatment effects available from each laser beam.

Another aspect of the aforementioned conventional art is mentioned below. When the irradiation pattern is switched over by use of the coupler or diffraction element, the irradiation pattern is determined by the coupler or diffraction element. Thus, unless a plurality of couplers or diffraction elements are used, the number of irradiation pattern variations could not be increased. It is therefore difficult to sufficiently enhance the treatment effects.

This disclosure is directed to provide an ophthalmic laser treatment apparatus capable of appropriately changing between irradiation patterns of laser beams to be irradiated from a tip of a probe.

An ophthalmic laser treatment apparatus provided as a typical aspect is an ophthalmic laser treatment apparatus for irradiating a laser beam to a patient's eye from a tip of a probe, including: a probe connector to which rear ends of a plurality of fibers of the probe are connected; a splitting unit for splitting a laser beam generated by a laser source into a plurality of laser beams; and a control unit for controlling the ophthalmic laser treatment apparatus, wherein the control unit is configured to: receive a command to select number of laser beams to be irradiated from the tip of the probe; drive the splitting unit according to the received command representing the number of laser beams to switch number of delivered beams corresponding to number of fibers to which the laser beam generated by the laser source will be delivered, of the plurality of fibers of the probe connected to the probe connector, and increase output of the laser source when the number of delivered beams is switched to an increased number and decrease the output of the laser source when the number of delivered beams is switched to a decreased number.

An ophthalmic laser treatment apparatus provided as another typical aspect is an ophthalmic laser treatment apparatus for irradiating a laser beam to a patient's eye from a tip of a probe, including: a probe connector to which rear ends of a plurality of fibers of the probe are connected; a deflecting unit for deflecting a laser beam generated by a laser source; and a control unit for controlling the ophthalmic laser treatment apparatus, wherein the control unit is configure to: receive a command to select an irradiation pattern of the laser beam to be irradiated from the tip of the probe; and control driving of the deflecting unit according to the received selection command to switch the fiber to which the laser beam will be delivered, of the plurality of fibers of the probe connected to the probe connector.

### BRIEF DESCRIPTION OF THE DRAWINGS

FIG. 1 is a perspective view showing a schematic configuration of an ophthalmic laser treatment apparatus;

FIG. 2 is a diagram showing one example of an operation screen displayed on a display part in a first embodiment;

FIG. 3 is a diagram showing a control system and optical systems of the ophthalmic laser treatment apparatus in the first embodiment;

FIG. 4 is a diagram showing an optical system of a splitting unit in the first embodiment;

FIG. 5 is an enlarged side view of a probe connector and a bundled fiber;

FIG. 6 is a flowchart of main processing to be executed by a control unit in the first embodiment;

FIG. 7 is a flowchart of setting change processing to be executed in the main processing in the first embodiment;

FIG. 8 is a diagram showing a control system and optical systems of an ophthalmic laser treatment apparatus in a second embodiment;

FIG. 9 is an explanatory view showing comparison in the number of fibers, fiber arrangement, and irradiation pattern of a probe per gauge;

FIG. 10 is a flowchart of main processing to be executed by a control unit in the second embodiment;

FIG. 11 is a flowchart of calibration processing to be executed in the main processing in the second embodiment; and

FIG. 12 is a flowchart of light emission processing to be executed in the main processing in the second embodiment.

### DETAILED DESCRIPTION OF THE EXEMPLARY EMBODIMENTS

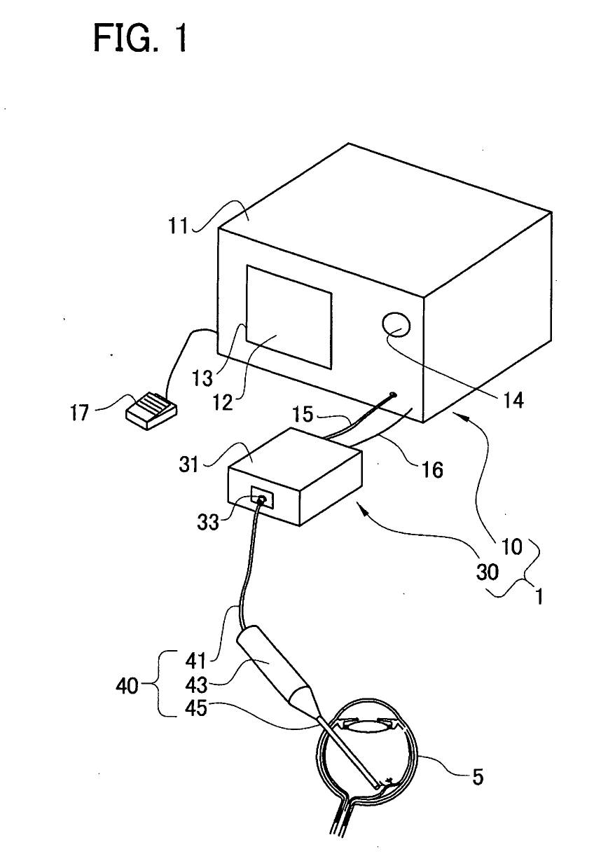

A first embodiment which is one of typical embodiments of this disclosure will be explained below referring to FIGS. 1 to 7. A schematic configuration of an ophthalmic laser treatment apparatus 1 in the first embodiment is first explained. In FIG. 1, an obliquely left lower side in FIG. 1 is assumed as the front side of the ophthalmic laser treatment apparatus 1. An obliquely right upper side in FIG. 1 is assumed as the rear side of the ophthalmic laser treatment apparatus 1. An obliquely left upper side in FIG. 1 is assumed as the left side of the ophthalmic laser treatment apparatus 1. An obliquely right lower side in FIG. 1 is assumed as the right side of the ophthalmic laser treatment apparatus 1. As shown in FIG. 1, the ophthalmic laser treatment apparatus 1 includes a main unit 10 and a splitting unit 30.

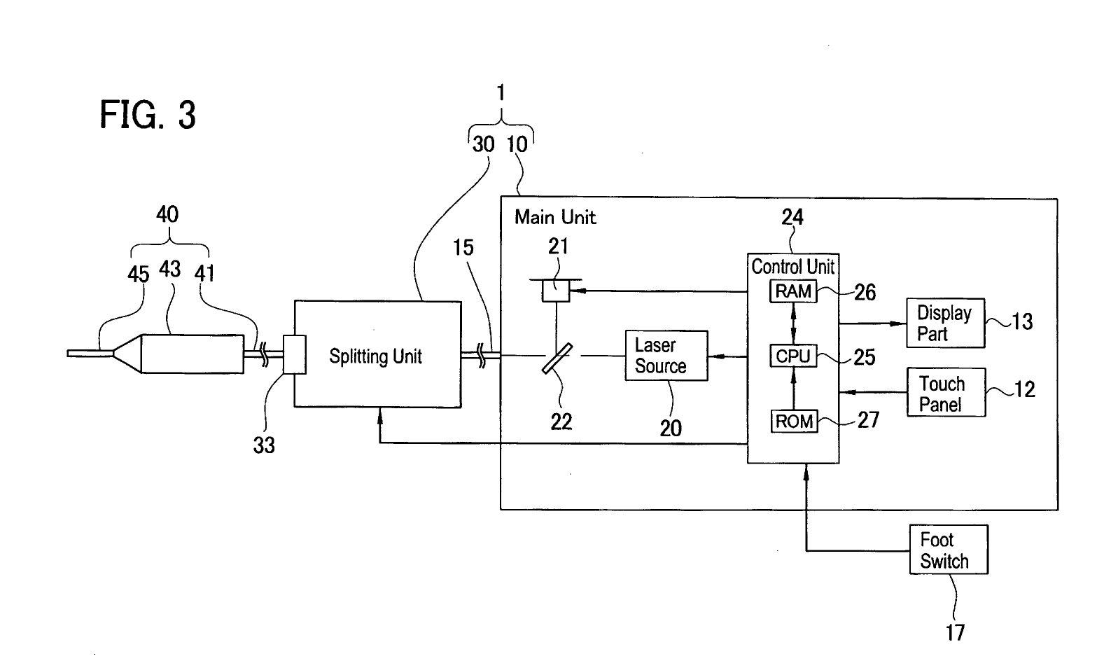

The main unit 10 includes a box-shaped casing 11 in which a laser source 20 (see FIG. 3) for generating a laser beam and others are housed. The main unit 10 is adapted to deliver a laser beam generated by the laser source 20 according to a set value to the splitting unit 30. The casing 11 is provided with a display part 13 on the front. The display part 13 is provided with a touch panel 12 on the front. When an operator touches some portions on the touch panel 12 corresponding to various buttons (the details will be mentioned later) displayed on the display part 13, various operation commands are input to the ophthalmic laser treatment apparatus 1.

The casing 11 is provided, at a right upper corner area of the front surface, with a power source button 14. The operator operates the power source button 14 to switch on or off the power of the ophthalmic laser treatment apparatus 1. To a right lower corner area of the front surface of the casing 11, a unit connecting fiber 15 and a cable 16 are connected. The unit connecting fiber 15 is connected between the main unit 10 and the splitting unit 30. The unit connecting fiber 15 delivers a laser beam generated by the laser source 20 (see FIG. 3) of the main unit 10 to the splitting unit 30. The cable 16 electrically connects the main unit 10 and the splitting unit 30. Furthermore, the main unit 10 is connected to a foot switch 17. When depressed by the operator, the foot switch 17 outputs a signal to generate a laser beam to the ophthalmic laser treatment apparatus 1.

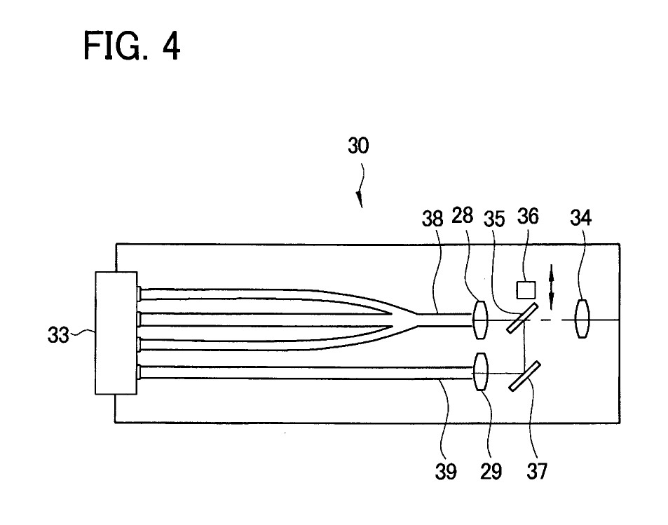

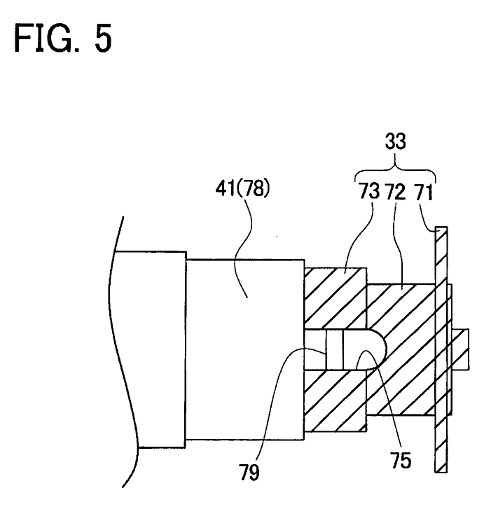

The splitting unit 30 includes a box-shaped casing 31 in which a system for splitting the laser beam delivered therein through the unit connecting fiber 15 into a plurality of laser lights (laser beams, laser light flux). To a back surface of the casing 31, the unit connecting fiber 15 and the cable 16 are connected. The casing 31 is provided with a probe connector 33 on the front. The probe connector 33 is a connector for connection with a probe (ENDO photo probe) 40. The operator is allowed to replace the probe 40 connected to the probe connector 33 in order to ensure cleanliness of the probe 40 or other purposes. The details of the system built in the splitting unit 30 and the details of the probe connector 33 will be described later referring to FIGS. 4 and 5.

The probe 40 is explained. The probe 40 includes a bundled fiber 41, a handpiece 43, and a needle 45. The bundled fiber 41 is formed by binding a plurality of fibers for delivering the laser beams. A rear end of the bundled fiber 41 is connected to the probe connector 33, thereby connecting the probe 40 to the splitting unit 30. The handpiece 43 is a member having a nearly columnar outer shape and will be gripped by the operator. The bundled fiber 41 connected to the probe connector 33 extends from the splitting unit 30 to the rear end of the handpiece 43. The needle 45 has a cylindrical shape extending forward from a tip of the handpiece 43. The bundled fiber 41 extends through the inside of the handpiece 43 and the inside of the needle 45 to the vicinity of a tip portion of the needle 45. The laser beam delivered from the splitting unit 30 to the bundled fiber 41 is irradiated from the tip portion of the needle 45.

Even though the details are not illustrated, the tip portion of the needle 45 is provided with a lens. This lens serves to focus a laser beam irradiated from each of the plurality of fibers. Accordingly, the plurality of laser beams emitted from the plurality of fibers are less likely to overlap each other. This reduces the possibility that the plurality of laser beams are concentrated to cause damage on a patient's eye 5.

The probe 40 of the present embodiment is injected into the patient's eye 5 to irradiate the laser beams mainly to a retina of the eye 5. The needle 45 of the present embodiment is therefore formed to be very thin to prevent damage on the patient's eye 5. As one example, the gauge (size gauge) of the probe 40 used in the present embodiment is 25 G (the diameter of the needle 45 is 0.50 mm) or 23 G (the diameter of the needle 45 is 0.63 mm). However, the gauge of the probe 40 may be changed. It is to be noted that when the operator intends to make treatment of the patient's eye 5 by use of the ophthalmic laser treatment apparatus 1, he/she is requested to advance the treatment while checking a condition of the retina through a surgical microscope.

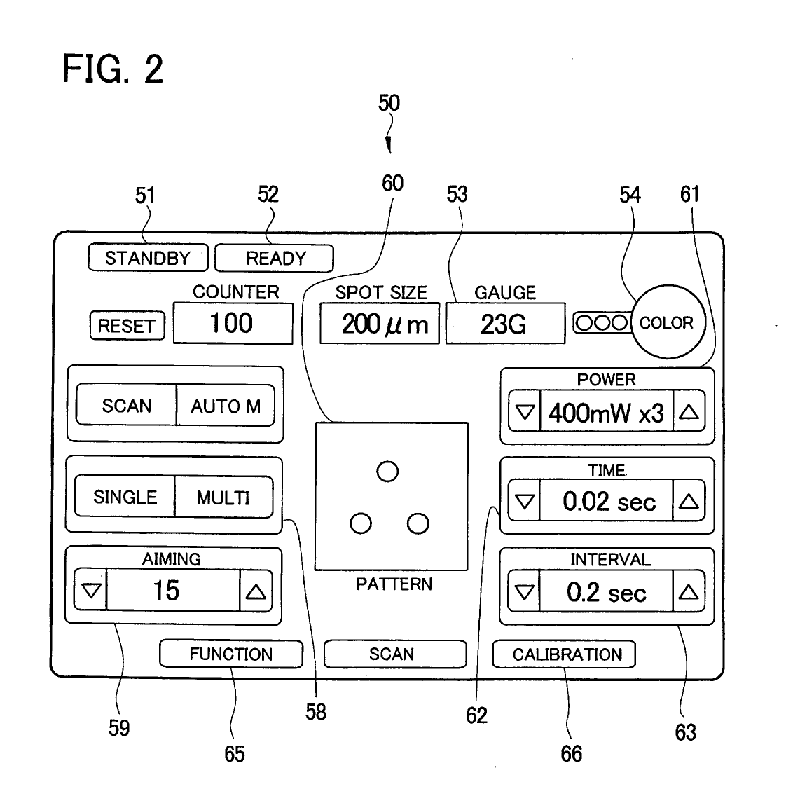

Referring to FIG. 2, one example of an operation screen 50 displayed on the display part 13 is explained. In an upper area of the operation screen 50, there are mainly displayed a standby button 51, a ready button 52, a gauge indicator part 53, and a laser beam selector part 54. The standby button 51 is operated to input a command to establish a standby mode in which the irradiation of a laser beam is disabled. The ready button 52 is operated to input a command to establish a ready mode in which the irradiation of a laser beam is enabled. The gauge indicator part 53 displays the gauge of the probe 40 connected to the probe connector 33 (see FIGS. 1, 3, and 4). The laser beam selector part 54 includes a button to receive a command to select any one of plural colors (e.g., red, yellow, and green) of laser beams and an indicator part to indicate a selected laser beam.

In a middle area of the operation screen 50 in an up-down direction, there are mainly displayed an irradiation pattern selector button 58, an aiming setting part 59, an irradiation pattern indicator part 60, a power setting part 61, an irradiation time setting part 62, and an interval setting part 63. The irradiation pattern selector button 58 is operated by the operator in selecting an irradiation pattern of a laser beam to be irradiated from a tip of the probe 40. In the first embodiment, there are provided an irradiation pattern to irradiate three laser beams so as to form apexes of a triangle and an irradiation pattern to irradiate a single laser beam. However, the irradiation patterns may be changed or modified appropriately. The aiming setting part 59 is used to set output of the aiming beam indicating an irradiation position of a laser beam. To be concrete, the aiming setting part 59 includes buttons to receive a command to set the output of the aiming beam and an indicator part to indicate the output of the aiming beam. The irradiation pattern indicator part 60 displays the selected irradiation pattern. The power setting part 61 includes buttons to receive a command to set the output of a laser beam to be irradiated from the tip of the probe 40, and an indicator part to indicate the set output of a laser beam. The irradiation time setting part 62 includes buttons to receive a command to set an irradiation time of a laser beam, and an indicator to indicate the set irradiation time. The irradiation time is a time from start to end of one irradiation of a laser beam. When the irradiation time is changed, the treatment effects vary. The interval setting part 63 includes buttons to set an interval (an irradiation interval) of continuous irradiation of laser beams and an indicator part.

In a lower area of the operation screen 50, a function button 65 and a calibration button 66 are displayed. The function button 65 is operated in carrying out various functions. For instance, when the probe 40 connected to the probe connector 33 is replaced with another probe 40 having a different gauge from the former, the operator inputs the gauge of the newly attached probe 40 in the ophthalmic laser treatment apparatus 1. In this case, the operator operates the function button 65 to display a gauge setting screen and input the gauge of the probe 40. The calibration button 66 is a button to input a command to carry out calibration. This calibration is a processing to deliver a laser beam to the probe 40, even though the details thereof will be explained in a second embodiment.

Referring to FIGS. 3 and 4, the control system and optical systems of the ophthalmic laser treatment apparatus 1 in the first embodiment will be explained below. As shown in FIG. 3, the main unit 10 of the ophthalmic laser treatment apparatus 1 includes the laser source 20, an aiming light source 21, a dichroic mirror 22, and a control unit 24.

The laser source 20 generates a laser beam to be used for treatment. The laser source 20 of the present embodiment can generate laser beams of red, yellow, and green. However, the laser source 20 has only to generate a laser beam appropriate for the treatment purpose and others. Thus, the laser source 20 may be changed to another. The aiming light source 21 generates an aiming beam. The aiming beam indicates an irradiation position of a laser beam to be irradiated from the tip of the probe 40. The dichroic mirror 22 is placed in the optical path of a laser beam and an aiming beam on the near side than the splitting unit 30. The dichroic mirror 22 reflects the aiming beam and transmits the laser beam. The laser beam generated by the laser source 20 passes through the dichroic mirror 22 and enters the unit connecting fiber 15. The aiming beam generated by the aiming light source 21 is reflected by the dichroic mirror 22 and enter, in a superimposed (that is, coaxial) state with the laser beam, into the unit connecting fiber 15.

The control unit 24 includes a CPU 25, a RAM 26 and a ROM 27. The CPU 25 is responsible for control of the ophthalmic laser treatment apparatus 1. The RAM 26 temporarily stores various data. The ROM 27 is a non-transitory storage media. The ROM 27 stores control programs, default values, and others for controlling operations of the ophthalmic laser treatment apparatus 1. The control unit 24 controls operations of the indicator part 13, laser source 20, aiming light source 21, splitting unit 30, and others based on signals input from the touch panel 12 and the foot switch 17 and others. The splitting unit 30 is operated under the control of the control unit 24 to switch the number of fibers (the number of delivered beams), through which the laser beam generated by the laser source 20 will be delivered, of the plurality of fibers included in the bundled fiber 41.

In the first embodiment, the bundled fiber 41 includes four fibers. Each entrance end of the four fibers is provided with a connector. In the probe connector 33 (see FIG. 5) of the splitting unit 30, four channels for emitting laser beams are provided. When the probe 40 is connected to the probe connector 33, the four connectors are respectively coincident in position with the four channels.

As shown in FIG. 4, the splitting unit 30 includes a collimator lens 34, an optical-path changing mirror 35, an optical-path changing drive part 36, a reflection mirror 37, condensing lenses 28 and 29, a fiber coupler 38, and a single-spot fiber 39. The collimator lens 34 converts the laser beam emitted from the unit connecting fiber 15 (see FIG. 3) into parallel light. The laser beam converted into the parallel light formed by the collimator lens 34 travels toward an entrance port of the fiber coupler 38. The optical-path changing mirror 35 is provided between the collimator lens 34 and the fiber coupler 38. The optical-path changing drive part 36 switches the position of the optical-path changing mirror 35 between a separated position located off an optical path extending from the collimator lens 34 to the fiber coupler 38 (hereinafter, referred to as an “optical path for multi-spot”) and a nonseparated position located on the multi-spot optical path. When the optical-path changing mirror 35 is placed on the separated position, the laser beam emitted through the collimator lens 34 travels on the multi-spot optical path without being reflected by the optical-path changing mirror 35, and then enters the entrance port of the fiber coupler 38. When the optical-path changing mirror 35 is placed on the nonseparated position, the laser beam emitted through the collimator lens 34 is reflected by the optical-path changing mirror 35 at a certain point of the multi-spot optical path, and further reflected by the mirror 37, then enters an entrance port of the single-spot fiber 39. The condensing lens 28 condenses the parallel light emitted through the collimator lens 34 to the fiber coupler 38. The condensing lens 29 condenses the parallel light emitted through the collimator lens 34 to the single-spot fiber 39.

The fiber coupler 38 of the first embodiment divides (branches) the laser light beam having entered in the single entrance port equally into three laser light beams. The fiber coupler 38 allows the divided three laser beams to come out one each from three emission ports. The single-spot fiber 39 allows the laser beam having entered in the entrance port to directly come out from a single emission port without being divided. The three emission ports of the fiber coupler 38 and the single emission port of the single-spot fiber 39 are connected one each to the four channels (not shown) of the back side (a right side in FIG. 4) of the probe connector 33. When the laser beam emitted from the collimator lens 34 enters in the fiber coupler 38, the laser beam is divided into three and then emitted from the three channels into the bundled fiber 41 (see FIGS. 1 and 3). On the other hand, when the laser beam enters in the single-spot fiber 39, the laser beam is emitted from the single channel into the bundled fiber 41 without being divided.

The probe connector 33 is explained in detail below referring to FIG. 5. In FIG. 5, a part with hatch lines is the probe connector 33. A remaining part with no hatch lines corresponds to a screw part 78 provided at a rear end of the bundled fiber 41. The probe connector 33 includes, from the order of a rear side (a right side in FIG. 5), a flange 71, a small-diameter part 72, and a large-diameter part 73. The flange 71 is a circular disc-like member. A back surface of the flange 71 is in contact with the casing 31 (see FIG. 1) of the splitting unit 30, thereby fixing the position of the probe connector 30 with respect to the housing 31. The small-diameter part 72 and the large-diameter part 73 are both nearly cylindrical members. The flange 71, small-diameter part 72, and large-diameter part 73 are arranged so that their central axes are coaxial. The diameter of the large-diameter part 73 is larger than the diameter of the small-diameter part 72. When the operator pushes the rear end of the bundled fiber 41 into the large-diameter part 73, the bundled fiber 41 comes to contact with a shoulder (not illustrated) formed between the small-diameter part 72 and the large-diameter part 73. As a result, the rear end of the bundled fiber 41 in an optical axis direction (a right-left direction in FIG. 5) of the laser beam is positioned in place. The large-diameter part 73 is formed with screw threads not shown on an outer peripheral surface of a leading end side (a left side in FIG. 5). The screw part 78 is provided to be rotatable with respect to the bundled fiber 41. The screw part 78 is formed with screw threads on an inner peripheral surface. While the bundled fiber 41 is inserted in the large-diameter part 73, when the screw part 78 is tightened with the screw threads of the large-diameter part 73, the probe 40 is connected (fixed) to the probe connector 33.

The probe connector 33 is provided with a positioning part 75 to make positioning of entrance ends of the plurality (four in the present embodiment) of fibers in predetermined places in the probe 40. As one example, the positioning part 75 of the first embodiment is a cutout formed from an end (a left end in FIG. 5) of the large-diameter part 73 to the small-diameter part 72. The outer peripheral surface of a part of the bundled fiber 41 to be inserted in the large-diameter part 73 is provided with a positioning key 79 protruding in a direction away from the axis. In a circumferential direction (an up-down direction in FIG. 5) of the bundled fiber 41, the length of the positioning key 79 is equal to the length of the positioning part 75. When the probe 40 is to be connected to the probe connector 33, the operator inserts the bundled fiber 41 in the large-diameter part 73 so that the positioning key 79 is inserted in the positioning part 75. Accordingly, the bundled fiber 41 is positioned in place with respect to the probe connector 33 in the circumferential direction of the bundled fiber 41. When the screw part 78 is tightened in this state, the bundled fiber 41 is connected so that the connectors of the predetermined fibers are appropriately positioned with respect to the four channels. Accordingly, the positioning part 75 contributes to avoid the occurrence of a defect that the laser beam is not delivered to a target fiber.

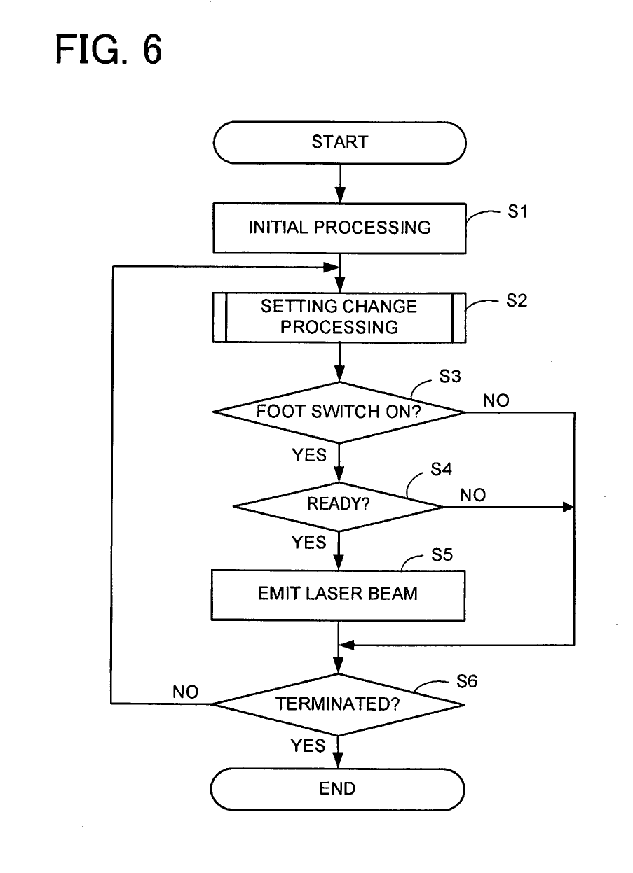

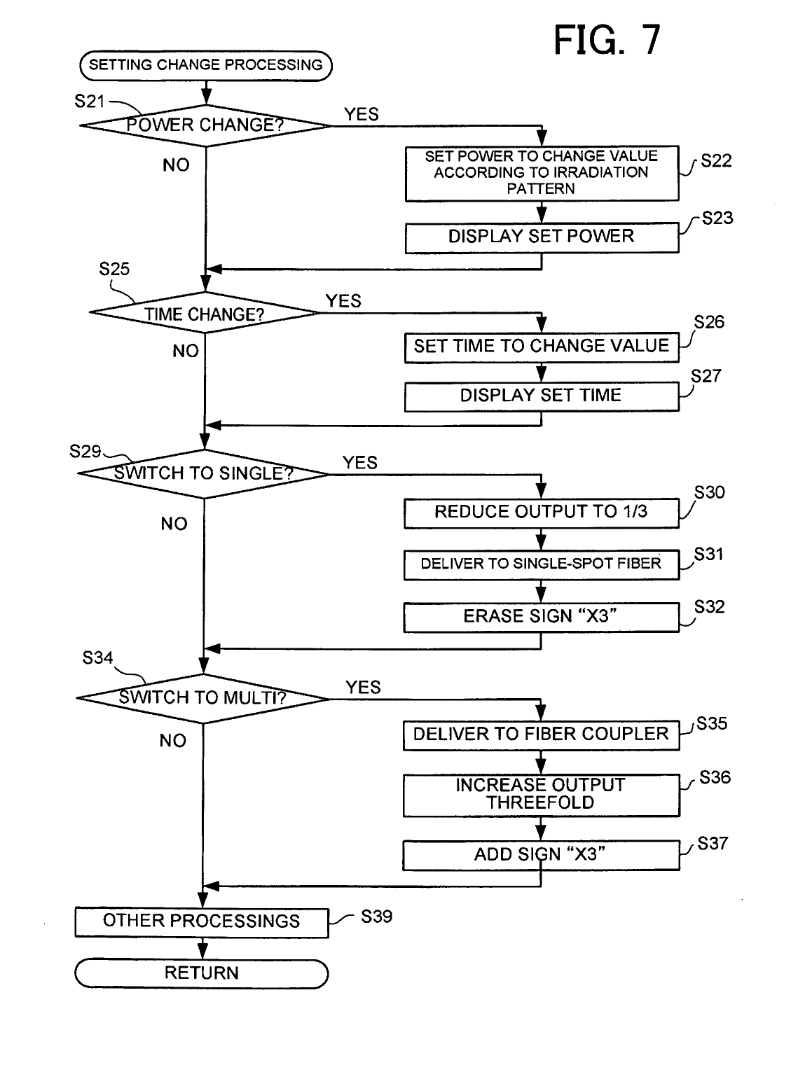

Referring to FIGS. 6 and 7, a main processing to be executed by the control unit 24 is explained below. The ROM 27 of the control unit 24 stores the control program for controlling operations of the ophthalmic laser treatment apparatus 1 as mentioned above. When the power of the ophthalmic laser treatment apparatus 1 is turned ON, the CPU 25 of the control unit 24 executes the main processing shown in FIG. 6 according to the control program.

Firstly, initial processing is performed (S1). In this initial processing, various processings are carried out, such as light emission of the aiming light source 21 (see FIG. 3), initial setting of an irradiation pattern (“SINGLE” or “MULTI”), initial setting of the power of the laser source 20, and initial setting of an irradiation time of the laser source. Secondly, setting change processing is performed (S2). In this setting change processing, processings to change settings such as power, irradiation time, and irradiation pattern are conducted. The details of the setting change processing will be explained later referring to FIG. 7.

Successively, it is determined whether or not the foot switch 17 (see FIGS. 1 and 3) has been turned ON (S3). If not turned ON (S3: NO), the processing directly advances to determination in S6. When the foot switch 17 is depressed and turned ON (S3: YES), it is determined whether or not a ready mode has been established (S4). If a standby mode, not the ready mode, has been established (S4: NO), the processing directly goes to S6. If the ready mode has been established (S4: YES), the laser source 20 emits a laser beam in the selected color with the output (power) and irradiation time set at that time (S5). If the set irradiation pattern is “MULTI”, the optical-path changing mirror 35 is driven to deliver the laser beam into the fiber coupler 38. Accordingly, three laser beams are simultaneously irradiated from the tip of the probe 40. On the other hand, if the set irradiation pattern is “SINGLE”, the optical-path changing mirror 35 is driven to deliver the laser beam into the single-spot fiber 39. Accordingly, a single laser beam is irradiated from the tip of the probe 40. It is then determined whether or not a command to terminate the processing has been input (S6). If not input (S6: NO), the processing returns to S2 and thus the processings in S2 to S5 are repeated. If the power supply is turned OFF and a termination command is input (S6: YES), the main processing is terminated.

The details of the setting change processing will be explained referring to FIG. 7. When the setting change processing is started, it is determined or not whether a command to change power is received (S21). The power change command is input by operation of a position on the touch panel 12 corresponding to the buttons of the power setting part 61 (see FIG. 2). If the power change command is not received (S21: NO), the processing directly goes to determination in S25. When the power change command is received (S21: YES), the output (power) of the laser source 20 is set according to the irradiation pattern set at that time (S22). The set output is displayed on the indicator part of the power setting part 61 (S23). The processing goes to the determination in S25.

In the present embodiment, to be concrete, the power to be set by use of the touch panel 12 by the operator is the power of the single laser beam or each of the plurality of laser beams to be irradiated from the tip of the probe 40. The same applies to the numeral value displayed on the power setting part 61. The control unit 24 sets the output of the laser source 20 so that the laser beam or each laser beam is irradiated with the output set by the operator from the probe 40. Accordingly, when the “SINGLE” irradiation pattern has been set, the control unit 24 sets the output of the laser source 20 based on the output set by the power setting part 61 directly to. When the “MULTI” irradiation pattern has been set, on the other hand, in consideration of the laser beam being to be divided into three, the control unit 24 sets the output of the laser source 20 as being three times larger than the output set by the power setting part 61. While the “MULTI” irradiation pattern is set, the control unit 24 further causes the indicator part of the power setting part 61 to display a sign “×3” which represents that three laser beams are irradiated with the set output from the probe 40. Accordingly, the operator can easily recognize the number of laser beams to be irradiated and the output of the laser source (the output three times larger than the set output in the present embodiment).

It is subsequently determined whether or not a command to change the irradiation time has been received (S25). The command to change the irradiation time is input by operating a position on the touch panel 12 corresponding to the buttons of the irradiation time setting part 62 (see FIG. 2). If the irradiation time change command has not been received (S25: NO), the processing directly advances to determination in S29. When the irradiation time change command has been received (S25: YES), the irradiation time is set to a commanded change value (S26). The set irradiation time is displayed on the indicator part of the irradiation time setting part 62 (S27). The processing goes to determination in S29.

Successively, it is determined whether or not the irradiation pattern has been switched from “MULTI” to “SINGLE” (S29). Changeover from “MULTI” to “SINGLE” is performed by operating a position on the touch panel 12 corresponding to the irradiation pattern selector button 58 (see FIG. 2). If not switched to “SINGLE” (S29: NO), the processing goes to determination in S24. When a command to switch to “SINGLE” is received (S29: YES), firstly, the output of the laser source 20 is reduced to one third (S30). Specifically, as the number of delivered beams, which is the number of laser beams, is set to N times (one third times in the present embodiment), the output of the laser source 20 is set to N times (one third times in the present embodiment). Consequently, the output of each laser beam irradiated from the probe 40 is equal before and after the number of delivered beams is changed.

After reduction of the output of the laser source 20 is completed, the optical-path changing drive part 36 (see FIG. 4) is controlled to drive the optical-path changing mirror 35 to be inserted onto the optical path of the laser beam. Thus, the laser beam output from the laser source 20 is delivered to the single-spot fiber 39 (see FIG. 4) (S31). Specifically, the laser beam is delivered to the probe 40 without being divided by the fiber coupler 38. Since the number of delivered beams is reduced after reduction of the output of the laser source 20 is completed, a risk that an intense laser beam generated from the laser source 20 with the output before reduction is directly irradiated from the probe 40 is largely reduced. The sign “×3” is erased from the power setting part 61 (S32), the processing goes to determination in S34. The control unit 24 does not change the irradiation time set in S26 and does keep this irradiation time constant even in the course (S29 to S32) of changing the irradiation pattern to “SINGLE”. This further restrain the variation in treatment effect due to switching of irradiation patterns (number of delivered beams).

Subsequently, it is determined whether or not the irradiation pattern has been switched from “SINGLE” to “MULTI” (S34). If it has not been switched (S34: NO), the processing directly goes to S39. If a command to switch to “MULTI” is received (S34: YES), the laser beam generated from the laser source 20 is firstly delivered to the fiber coupler 38 (see FIG. 4) (S35). Specifically, the optical-path changing drive part 36 is controlled to drive to move the optical-path changing mirror 35 out of the optical path of the laser beam. The laser beam generated from the laser source 20 is thus divided by the fiber coupler 38 and then delivered to the probe 40.

After completion of switching of the number of delivered beams by the fiber coupler 38, the output of the laser source 20 is increased (S36). Specifically, as the number of delivered beams is set to N times (three times in the present embodiment), the output of the laser source 20 is set to N times (three times in the present embodiment). Consequently, the output of each laser beam irradiated from the probe 40 is equal before and after the number of delivered beams is changed. The sign “×3” representing the irradiation pattern of irradiating three laser beams is added in the power setting part 61 (S37) and the processing goes to S39. Then, other processings are conducted (S39) and the flow returns to the main processing (see FIG. 6). In the processing in S39, for example, the processing of switching between the standby mode and the ready mode, the processing of changing the setting of interval, and others are performed.

The ophthalmic laser treatment apparatus 1 of the first embodiment can switch the number of fibers (the number of delivered beams) of the probe 40 to which the laser beam generated by the laser source 20 is to be delivered, as explained above. Accordingly, the number of laser beams to be irradiated from the tip of the probe 40 is switched. Thus, the operator can easily switch the irradiation pattern of the laser beam to another without replacing the probe 40 in use to efficiently make treatment. The ophthalmic laser treatment apparatus 1 is further arranged to increase the output of a laser beam as the number of delivered beams is increased or decrease the output of the laser beam as the number of delivered beams is reduced. Therefore, an energy change amount of each laser beam irradiated from the probe 40 is made smaller than that in a case where the output of a laser beam is not adjusted. The ophthalmic laser treatment apparatus 1 of the first embodiment can therefore switch the irradiation pattern of a laser beam to be irradiated from the tip of the probe 40 while restraining variation in treatment effects available from each laser beam.

The ophthalmic laser treatment apparatus 1 of the first embodiment is arranged to adjust the output of the laser source 20 itself without using an attenuator or the like for attenuating the output of a laser beam, thereby adjusting the output of each laser beam to be irradiated. Even when the number of delivered beams is reduced, therefore, the energy (concretely, electric power) used for treatment is not wasted. The ophthalmic laser treatment apparatus 1 of the first embodiment is also arranged to select whether or not the laser beam is to be divided without using a system for deflecting the laser beam, to switch the number of laser beams to be irradiated from the probe 40. Consequently, the ophthalmic laser treatment apparatus 1 of the first embodiment can switch between the irradiation patterns with a simple configuration while restraining variation in treatment effects.

The treatment effects vary depending on the irradiation time of a laser beam. In this case, the operator has to change the treatment method or the like in light of treatment effects which may vary. For instance, if it is assumed that the output of a laser beam is P times and the irradiation time is 1/P times, the energy needed for irradiation of the laser beam is constant, but the thermal influence of the laser beam on eye tissues and others is different, resulting in nonconstant treatment effects. The ophthalmic laser treatment apparatus 1 of the first embodiment can adjust the output of a laser beam while maintaining the irradiation time, thereby further restraining variation in treatment effect.

In the ophthalmic laser treatment apparatus 1 of the first embodiment, when the number of delivered beams is set to N times, the output of the laser source 20 is set to N times. Consequently, the energy of each laser beam to be irradiated from the probe 40 is constant before and after the number of delivered beams is switched to another. Accordingly, the ophthalmic laser treatment apparatus 1 can further restrain variation in treatment effects which may be caused when the number of delivered beams is switched to another.

The probe connector 33 of the first embodiment includes the positioning part 75 for positioning entrance ends of the plurality of fibers at predetermined positions. Thus, the laser beams divided by the splitting unit 30 are allowed to reliably enter in corresponding entrance ends positioned by the positioning part 75. Specifically, in the ophthalmic laser treatment apparatus 1, the positioning part 75 can reduce the possibility of the occurrence of a defect that a laser beam is not delivered to a target fiber. This can prevent changes in energy of each laser beam. For instance, even when the probe 40 is replaced with another to ensure cleanliness of the probe 40 and other purposes, the ophthalmic laser treatment apparatus 1 can easily restrain changes in energy of each laser beam to be irradiated.

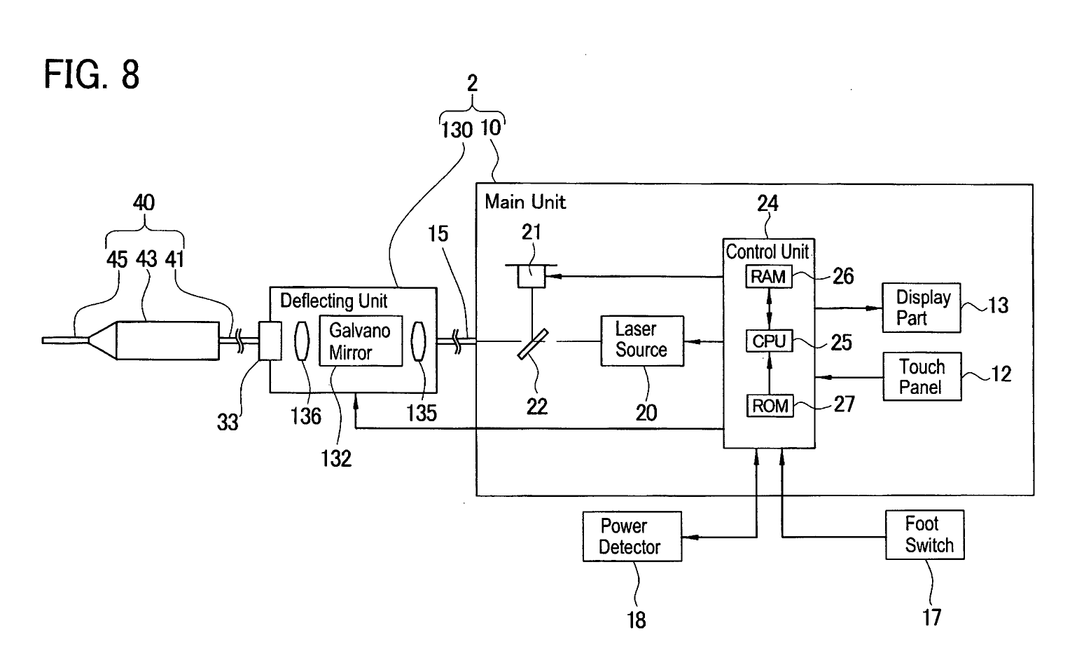

A second embodiment which is one of typical embodiments of this disclosure will be explained referring to FIGS. 8 to 12. An ophthalmic laser treatment apparatus 2 of the second embodiment differs from that of the first embodiment in that a deflecting unit 130 is provided instead of the splitting unit 30 of the first embodiment. A schematic configuration of the second embodiment excepting the deflecting unit 130 may be common to the schematic configuration (see FIG. 1) of the first embodiment. Part of the display contents on the operation screen 50 appearing on the display part 13 shown in FIG. 8 is also common to the display contents on the operation screen (see FIG. 2) of the first embodiment. The control system and the optical systems shown in FIG. 8 are also partially common to the control system and the optical systems (see FIG. 3) of the first embodiment. The ophthalmic laser treatment apparatus 2 of the second embodiment includes identical configurations to the probe connector 33 and the bundled fiber 41 (see FIG. 5) of the first embodiment. Therefore, the explanation of the second embodiment is given with the same reference signs to the configurations common to those in the first embodiment. Their explanations are omitted or simplified.

The control system and the optical systems of the ophthalmic laser treatment apparatus 2 of the second embodiment will be explained below referring to FIG. 8. The main unit 10 of the ophthalmic laser treatment apparatus 2 includes the laser source 20, the aiming light source 21, the dichroic mirror 22, and the control unit 24. The control unit 24 is connected to the foot switch 17 and a power detector 18.

The power detector 18 is a device to be used in executing calibration. This calibration, the details of which will be mentioned later, is executed by causing the laser source 20 to generate a laser beam and detecting the output of the laser beam irradiated from the tip of the probe 40. The power detector 18 includes a needle insertion port and an output detecting part. In the needle insertion port, a tip portion of the needle 45 of the probe 40 is inserted. Since the needle 45 is inserted in the needle insertion port, outward leakage of a laser beam can be prevented. The output detecting part detects the output of the laser beam irradiated from the tip of the needle 45 inserted in the needle insertion port. The power detector 18 transmits a detection result from the output detecting part to the control unit 24.

The deflecting unit 130 of the ophthalmic laser treatment apparatus 2 includes the probe connector 33, a galvano mirror 132, a collimator lens 135, and a condensing lens 136. This probe connector 33 may also be configured as with the probe connector 33 of the first embodiment. The galvano mirror 132 deflects (scans in the present embodiment) a laser beam delivered thereto through the unit connecting fiber 15. The collimator lens 135 converts the laser beam emitted from the unit connecting fiber 15 into parallel light to make the parallel light fall on the galvano mirror 132. The condensing lens 136 condenses the incoming laser beam from the galvano mirror 132, and delivers the laser beam to the bundled fiber 41 connected to the probe connector 33.

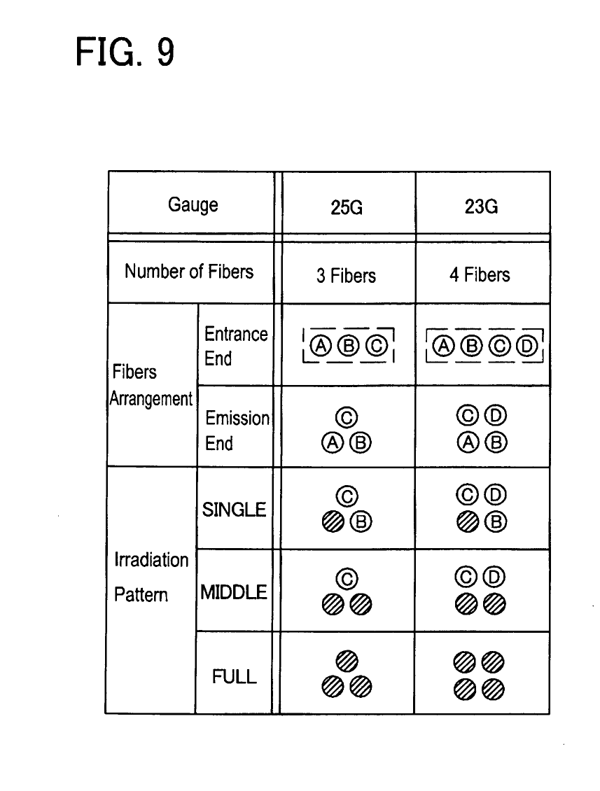

Referring to FIG. 9, the number of fibers, fiber arrangement, and irradiation patterns of the probe 40 of the second embodiment will be explained below. The number of fibers is the number of fibers included in the bundled fiber 41 of the probe 40. In the present embodiment, as one example, the probe 40 of 25 gauge (the diameter of the needle 45 is 0.50 mm) includes three fibers. The probe 40 of 23 gauge (the diameter of the needle 45 is 0.63 mm) includes four fibers. However, the gauge of the probe 40, the number of fibers of the probe 40, and others may be changed appropriately. For example, the number of fibers may be increased by decreasing the diameter of each fiber without changing the gauge.

The fiber arrangement is explained. In the probe 40 of the second embodiment, the arrangement of entrance ends and the arrangement of emission ends of the plurality of fibers are different from each other. The entrance ends are end portions on the side in which laser beams enter from the deflection unit 130 (that is, end portions on a rear end side connected to the probe connector 33). The emission ends are end portions on the leading end side from which laser beams are irradiated. In the probe 40 of 25 gauge, the entrance ends of three fibers A, B, and C are arranged in linear array when seen in an optical axis direction of the laser beam having passed through the collimator lens 135 (see FIG. 8). Similarly, in the probe 40 of 23 gauge, the entrance ends of four fibers A, B, C, and D are also arranged in linear array when seen in the optical axis direction of a laser beam. On the other hand, in the probe 40 of 25 gauge, the emission ends of the three fibers A, B, and C are arranged in a triangular form. In the probe 40 of 23 gauge, the emission ends of the four fibers A, B, C, and D are arranged in a rectangular form.

To the probe connector 33, the probe 40 is connected with the entrance ends of the plurality of fibers being arranged in line. The ophthalmic laser treatment apparatus 2 therefore can selectively deliver the laser beam to each of the plurality of fibers by scanning, in one direction, the laser beam generated by the laser source 20. The probe 40 of the second embodiment in which the arrangement of the entrance ends and the arrangement of the emission ends of the fibers are made different from each other can simplify the structure and control to deflect (scan) a laser beam and also can irradiate the laser beam from the tip in various patterns.

The diameter of each fibers used in the probe 40 of the present embodiment is smaller in the emission ends than in the entrance ends. To be concrete, each fiber of the present embodiment is a tapered fiber having the diameter gradually decreasing from an entrance end to an emission end. Accordingly, this can reduce the possibility that the laser beam emitted from the galvano mirror 132 is not appropriately delivered to each fiber and also can easily produce the needle 45 having a thin diameter. However, the probe 40 also may be configured without using the tapered fibers.

Furthermore, the emission end (the tip) of each fiber is formed in a tapered shape so that the inner peripheral surface of each fiber is exposed to the outside of the needle 45. Thus, the laser beam irradiated from each fiber is allowed to slightly spread toward the outside of the needle. The probe 40 therefore can reduce the possibility that the plurality of laser beams irradiated from the emission ends of the plurality of fibers are superimposed one on another. This can reduce the possibility that the plurality of laser beams are concentrated, leading to damage to the patient's eye 5. A configuration that each of the emission ends of the fibers is bent toward the outside of the needle 45 can also reduce the possibility that the plurality of laser beams are superimposed.

Even though the details are not illustrated, the tip of the needle 45 is provided with a lens for condensing a laser beam irradiated from each of the plurality of fibers. Accordingly, the possibility that the plurality of laser beams are dispersed and thus superimposed is further reduced.

Even though the details are not illustrated, furthermore, the entrance end of each of the plurality of fibers of the probe 40 is provided with a connector. In the probe connector 33 (see FIG. 5) of the deflecting unit 130, four channels A, B, C, and D are provided, to which laser beam deflected by the galvano mirror 132 are delivered. When the 25-gauge probe 40 is connected to the probe connector 33, the fiber A is connected to the channel A, the fiber B is connected to the channel B, and the fiber C is connected to the channel C, respectively, through the connectors. In this case, the channel D is not used. When the 23-gauge probe 40 is connected to the probe connector 33, the fiber A is connected to the channel A, the fiber B is connected to the channel B, the fiber C is connected to the channel C, and the fiber D is connected to the channel D, respectively, through the connectors.

The irradiation pattern is explained. The irradiation pattern is an irradiation pattern of a laser beam to be irradiated from the tip of the probe 40. In the second embodiment, one of three irradiation patterns, “SINGLE”, “MIDDLE”, and “FULL” is selectable by the operator. In a case where the “SINGLE” pattern is selected, the ophthalmic laser treatment apparatus 2 controls the galvano mirror 132 to deliver the laser beam to the channel A (i.e., the fiber A). Consequently, the irradiation pattern becomes an irradiation pattern in which a single laser beam is irradiated irrespective of the gauge of the probe 40. In the present embodiment, specifically, the “SINGLE” pattern for single spot irradiation is included in the plurality of irradiation patterns.

In a case where the “MIDDLE” pattern is selected, the ophthalmic laser treatment apparatus 2 causes the galvano mirror 132 to scan the laser beam so that the laser beam is delivered to the channels A and B (i.e., the fibers A and B) in order. Consequently, the irradiation pattern becomes an irradiation pattern in which two laser beams are irradiated irrespective of the gauge of the probe 40.

In a case where the “FULL” pattern is selected and the 25-gauge probe 40 is being used, the ophthalmic laser treatment apparatus 1 delivers the laser beam to the three channels A, B, and C. In this case, the irradiation pattern is an irradiation pattern to irradiate three laser beams so as to form the apexes of a triangle. When the “FULL” pattern is selected and the 23-gauge probe 40 is being used, the ophthalmic laser treatment apparatus 2 delivers the laser beam to the four channels A, B, C, and D. In this case, the irradiation pattern is an irradiation pattern to irradiate four laser beams so as to form the apexes of a rectangle. As above, the ophthalmic laser treatment apparatus 2 can change the irradiation pattern to another by driving the galvano mirror 132 of the deflecting unit 130 to deflect (scan) the laser beam. It is further possible to appropriately drive the galvano mirror 132 according to the probe connected to the probe connector 33.

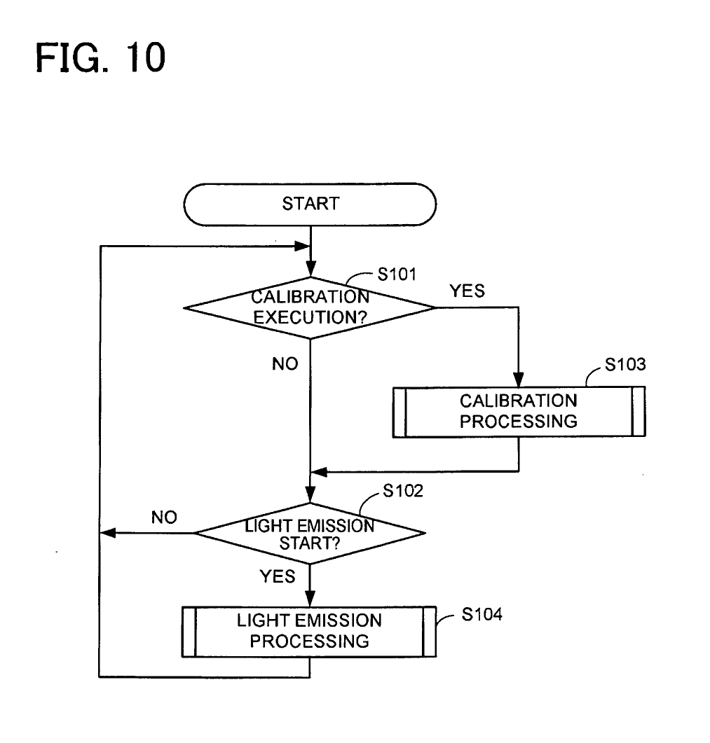

Referring to FIGS. 10 to 12, main processing to be executed by the control unit 24 of the second embodiment is explained. The ROM 27 of the control unit 24 stores control programs to control operations of the ophthalmic laser treatment apparatus 2. When the power source of the ophthalmic laser treatment apparatus 2 is turned on, the CPU 25 of the control unit 24 executes the main processing shown in FIG. 10 according to the control program.

It is first determined whether or not the timing to execute calibration has come (S101). In the present embodiment, when a position on the touch panel 12 corresponding to the calibration button 66 of the operation screen 50 (see FIG. 2) is operated, it is determined that the timing to execute calibration has come. However, the timing to execute calibration may be changed appropriately. For instance, a switch for detecting that the probe 40 has been connected to the probe connector 33 may be provided. In this case, the CPU 25 may use the timing at which the connection of the probe 40 is detected by a switch as the calibration execution timing.

When the calibration execution timing has not come (S101: NO), it is determined whether or not a command to start emission of a laser beam (i.e., treatment) has issued (S102). In the present embodiment, the touch panel 12 is operated to input a command to start the emission and a command to terminate the emission. When the emission start command is not issued (S102: NO), the processing directly returns to determination in S101, and then determinations in S101 and S102 are repeated.

When the calibration execution timing has come (S101: YES), the calibration is performed (S103) and the processing goes to determination in S102. When the emission start command is issued (S102: YES), the emission processing is performed (S104) and the processing returns to determination in S101. When the power source of the ophthalmic laser treatment apparatus 2 is turned off, the main processing shown in FIG. 10 is terminated.

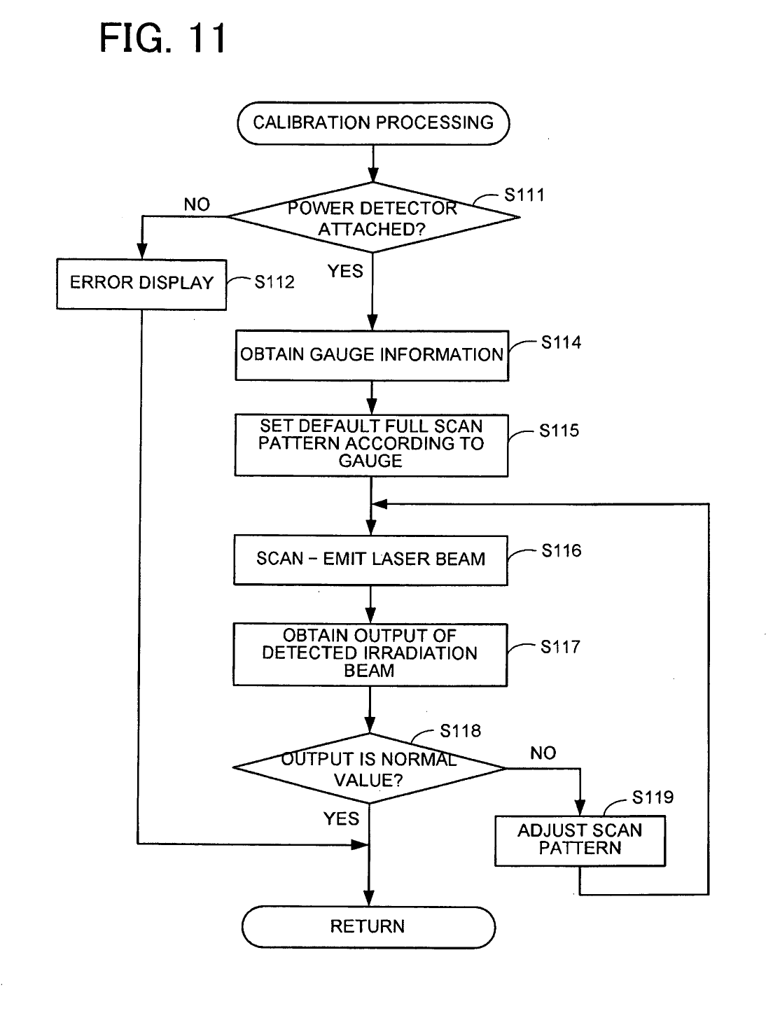

Referring to FIG. 11, the details of the calibration processing is explained. When the calibration processing is started, it is determined whether or not the power detector 18 (see FIG. 8) has been attached to the probe 40 (S111). Specifically, it is determined whether or not the needle 45 has been inserted in the needle insertion port of the power detector 18. When a predetermined time has passed while the power detector 18 is not attached (S111: NO), the display part 13 displays an error indication to inform an operator of that the calibration is not executable (S112), and the calibration processing is terminated.

When the power detector 18 is attached (S111: YES), information on the gauge of the probe 40 connected to the probe connector 33 is obtained (S114). As one example, in the present embodiment, the gauge information is set in advance by operation of the touch panel 12 by the operator. Subsequently, a FULL scan pattern which is a default according to the gauge (25 G or 23 G in the present embodiment) is set as a scan pattern for calibration of the galvano mirror 132 (S115). To be specific, the FULL scan pattern for 25 gauge is a scan pattern to deliver a laser beam to the channels A, B, and C in this order. The FULL scan pattern for 23 gauge is a scan pattern to deliver a laser beam to all of the channels A, B, C, and D. The default scan pattern represents a scan pattern set when a normal probe 40 is connected to a correct position of the probe connector 33. Specifically, if no defects such as connection failure of the probe 40 are found, the galvano mirror 132 is driven for scanning in the default scan pattern to accurately deliver a laser beam to the plurality of fibers of the probe 40.

The CPU 25 then causes the galvano mirror 132 in the set scan pattern and makes the laser source 20 emit light at set timings (S116). The output of the laser beam detected by the output detecting part of the power detector 18 is obtained (S117). It is determined whether or not the detected output is a normal value (S118). If it is not a normal value (S118: NO), the entrance ends of the plurality of fibers of the probe 40 may not be positioned in correct places. Thus, the scan pattern is adjusted (S119) and the processings in S116 to S118 are performed again. If the detected output is a normal value (S118: YES), the processing returns to the main processing. If the normal output is not detected even though the processings in S116 to S118 have been repeated a predetermined number of times, the error display is made to appear and the processing returns to the main processing.

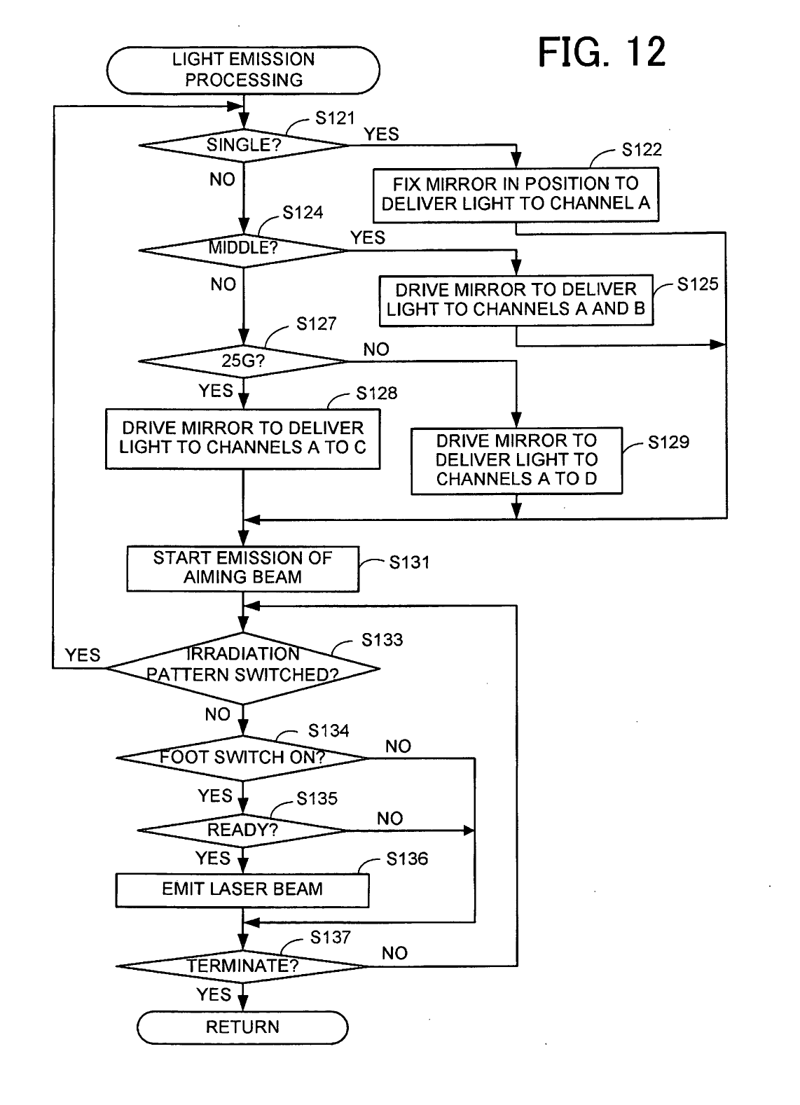

Referring to FIG. 12, the emission processing is explained. When the emission processing is started, it is determined whether or not the irradiation pattern selected by the selection command received through the touch panel 12 is “SINGLE” (S121). If the “SINGLE” pattern is selected (S121: YES), the galvano mirror 132 is fixed in a position to deliver the laser beam generated by the laser source 20 to the channel A (S122). The processing then goes to S131.

If the irradiation pattern is not “SINGLE” (S121: NO), it is determined whether or not the selected irradiation pattern is “MIDDLE” (S124). If the “MIDDLE” pattern is selected (S124: YES), the galvano mirror 132 is driven to deliver the laser beam to the channels A and B of the four channels A, B, C, and D (S125). The processing then goes to S131.

If the irradiation pattern is “FULL”, neither “SINGLE” nor “MIDDLE” (S124: NO), it is determined whether or not the gauge of the probe 40 connected to the probe connector 33 is 25 G (S127). If the gauge has been set to 25 G by operation of the touch panel 12 (S127: YES), the galvano mirror 132 is driven to deliver the laser beam generated by the laser source 20 to the channels A, B, and C (S128). The processing then goes to S131. If the gauge of the probe 40 is 23 G not 25 G (S127: NO), the galvano mirror 132 is driven to deliver the laser beam to all of the channels A, B, C, and D (S129).

Successively, the aiming light source 21 starts to emit the aiming beam (S131). The aiming beam is irradiated to the same position as the position at which the laser beam will be irradiated. It is then determined whether or not a command to switch the irradiation pattern to another has been input (S133). In the present embodiment, as mentioned above, a position on the touch panel 12 corresponding to the irradiation pattern selector button 58 (see FIG. 2) is operated to input the command to switch the irradiation pattern to another. When the irradiation pattern switching command is input (S133: YES), the selected irradiation pattern is displayed on the irradiation pattern indicator part 60 (see FIG. 2) and then the processing returns to determination in S121. Thereafter, the galvano mirror 132 is driven according to the selected irradiation pattern (S121 to S129).

When the irradiation pattern switching command is not input (S133: NO), it is determined whether or not the foot switch 17 has been turned ON (S134). If not turned ON (S134: NO), the processing directly goes to determination in S137. When the foot switch 17 is depressed and turned ON (S134: YES), it is determined whether or not the ready mode has been established (S135). When the standby mode has been established, not the ready mode (S135: NO), the processing directly goes to determination in S137. When the ready mode has been established (S135: YES), the laser beam of a selected color is emitted by the laser source 20 at the timing set in advance according to the irradiation pattern (S136). To be specific, the galvano mirror 132 is placed in a rest state for a period longer than the set irradiation time every time when the laser beam is delivered to the target channel. After the rest time has passed, the galvano mirror 132 is changed to another angle to deliver the laser beam to a next target channel. The control unit 24 emits the laser beam only for the set irradiation time every time when the galvano mirror 132 comes to the rest state. Consequently, the laser beam is irradiated in the selected irradiation pattern from the probe 40. The rest time of the galvano mirror 132 has only to be equal to or longer than the set irradiation time. The rest time therefore may be set to a single fixed time irrespective of the irradiation time. It is then determined whether or not a command to terminate light emission has been input (S137). If not input (S137: NO), the processing returns to determination in S133 and the processings in S133 to S137 are repeated. When the termination command is input (S137: YES), the processing returns to the main processing (see FIG. 10).

In the ophthalmic laser treatment apparatus 2 of the second embodiment, as explained above, the fiber to which the laser beam will be delivered is switched to another of the plurality of fibers of the probe 40, thereby enabling changing between the irradiation patterns of the laser beam to be irradiated from the tip of the probe 40. This makes it easy for the operator to switch between the irradiation patterns of the laser beam without replacing the probe in use during surgery, thereby enabling effective treatment. In the ophthalmic laser treatment apparatus 2 of the second embodiment, furthermore, driving of the galvano mirror 132 is controlled to switch between the fibers to which the laser beam will be delivered. This can easily supply the laser beam in a number of variations to the fibers in an easier manner than in a case of using a coupler, diffraction element, and so on. In the ophthalmic laser treatment apparatus 2 of the second embodiment, therefore, variations of the irradiation positions of the laser beam to be irradiated from the tip of the probe 40 may be easily increased as compared with the case of using a coupler or a diffraction element and others. The ophthalmic laser treatment apparatus 2 is also arranged to deflect the laser beam generated by the laser source 20 to change the irradiation pattern to another. Even when the irradiation pattern is changed over, accordingly, the laser beam or each of the laser beams to be irradiated from the tip of the probe 40 does not change in output of each laser beam. Accordingly, even when the irradiation pattern is changed over, the treatment effects are remained.

The probe connector 33 of the second embodiment is connected with the probe 40 so that the entrance ends located at the rear end portion of the fibers are arranged on the same plane with the plane on which the laser beam will be scanned by the galvano mirror 132. In the second embodiment, in other words, the plurality of entrance ends are arranged in line when seen from the galvano mirror 132. Accordingly, the ophthalmic laser treatment apparatus 2 can simplify the configuration of a portion (the galvano mirror 132 in the present embodiment) for changing the laser beam and driving control thereof, thereby easily increasing the variations of laser beam irradiation positions.

The probe connector 33 of the second embodiment includes the positioning part 75 for positioning the entrance ends of the plurality of fibers at predetermined positions as in the first embodiment. Accordingly, the laser beam deflected by the deflecting unit 130 is easily made to reliably enter in each entrance end positioned by the positioning part 75. In the ophthalmic laser treatment apparatus 2, therefore, the positioning part 75 can reduce the possibility of the occurrence of a defect that the laser beam is not delivered to a target fiber, and variations of the irradiation positions of the laser beam can be increased.

In many cases, the gauge of the probe 40 is different according to the number of fibers in the probe 40, the diameter of each fiber, and others. The ophthalmic laser treatment apparatus 2 of the second embodiment arranged to appropriately operate according to the gauge of the connected probe 40 can appropriately increase irradiation position variations of the laser beam. The ophthalmic laser treatment apparatus 2 also can make calibration. Thus, even in a case where the probe 40 connected to the probe connector 33 is replaced with another one or in other cases, the ophthalmic laser treatment apparatus 2 can more reliably deliver the laser beam generated by the laser source 20 to a target fiber.

The aforementioned first and second embodiments are mere typical examples. Therefore, the above first and second embodiments may be modified.

In the ophthalmic laser treatment apparatus 1 of the first embodiment, for instance, the main unit 10 including the laser source 20 is separately provided from the splitting unit 30 for splitting a laser beam. Accordingly, a manufacturer that manufactures and sells the ophthalmic laser treatment apparatus 1 can produce and sell only the splitting unit 30, so that this splitting unit 30 is connected in use to the main unit 10 already possessed by an operator. In this case, the manufacturer may install a program to execute the main processing shown in FIG. 6 in the main unit 10 possessed by the operator, as needed. The control unit for controlling the splitting unit (the fiber coupler 38 in the above embodiments) for splitting a laser beam may also be provided in the splitting unit 30 instead of the main unit 10. Needless to say, the main unit 10 and the splitting unit 30 may be integrally formed, not separately. Similarly, the manufacturer can produce and sell only the deflecting unit 130 exemplified in the second embodiment, so that this deflecting unit 130 is connected in use to the main unit 10 already possessed by the operator. In this case, the manufacturer may install the program for executing the main processing shown in FIG. 10 in the main unit 10 possessed by the operator, as needed. The control unit may also be provided in the deflecting unit 130. The main unit 10 and the deflecting unit 130 also may be integrally formed.

In the first embodiment, the fiber coupler 38 (i.e., a fiber optical coupler) is used as the splitting unit for splitting (branching) the laser beam generated by the laser source 20. However, the structure of the splitting unit may be changed appropriately. For instance, at least any one of a DOE element for dividing a laser beam (beam), a waveguide optical coupler for equally distributing light, a diffracting grating for diffracting light, and others may be used as the splitting unit. In this case, the ophthalmic laser treatment apparatus 1 also has to connect the probe 40 so as to deliver a plurality of laser beams divided by the splitting unit to the plurality of fibers of the probe 40.

The ophthalmic laser treatment apparatus 1 of the first embodiment is arranged to switch whether or not the optical-path changing mirror 35 is inserted in the optical path of the laser beam, thereby changing the number of delivered beams. However, the method of changing the number of delivered beams may be changed. For instance, the ophthalmic laser treatment apparatus 1 may be arranged to move the splitting unit itself to switch between the presence and the absence of the splitting unit in the optical path of the laser beam, thereby changing the number of delivered beams.

In the ophthalmic laser treatment apparatus 1 of the first embodiment, the output of the laser source 20 is changed according to the number of delivered beams. The ophthalmic laser treatment apparatus 1 therefore can restrain variation in treatment effect without wasting electric power. However, the ophthalmic laser treatment apparatus 1 may be arranged to adjust the output of a laser beam by using another method. For instance, the ophthalmic laser treatment apparatus 1 may also be configured to insert or remove an attenuator or the like for attenuating the output of the laser beam in or from the optical path of the laser beam to adjust the output of the laser beam. In this case, it is possible to restrain variation in the treatment effect obtained by each laser beam per irradiation pattern. A deflecting plate may be rotated about an optical axis of a laser beam to adjust the output of the laser beam.

The ophthalmic laser treatment apparatus 1 of the first embodiment is arranged to switch between the irradiation pattern to irradiate three laser beams and the irradiation pattern to irradiate a single laser beam. However, it is obviously possible to appropriately change the kind of the irradiation pattern. For instance, the ophthalmic laser treatment apparatus 1 may include a plurality of splitting units different in the number of laser beams to be split and change between the splitting units to be inserted in the optical path of the laser beam to thereby switch between the irradiation patterns. In this case, the ophthalmic laser treatment apparatus 1 can change the number of laser beams to be irradiated in the “MULTI” pattern without switching to the “SINGLE” pattern and the “MULTI” pattern. Switching may also be conducted between three or more irradiation patterns.

The ophthalmic laser treatment apparatus 1 of the first embodiment is configured such that the output is set to N times when the number of delivered beams is set to N times and the irradiation time is maintained before and after the number of delivered beams is changed, so that the energy of each laser beam to be irradiated from the probe 40 is made constant. Accordingly, the variation in treatment effects is greatly restrained. However, the ophthalmic laser treatment apparatus 1 is configured to increase the output of the laser beam when the number of delivered beams is increased and decrease the output when the number of delivered beams is decreased, so that the variation in treatment effects can be restrained as compared with the case where the output is not adjusted. In other words, the same effects can be obtained even by different concrete output adjusting methods.

The first and second ophthalmic laser treatment apparatuses 1 and 2 receive various commands such as the selection command of splitting unit when the touch panel 12 provided on the surface of the display part 13 is operated. However, the method of receiving commands may be changed appropriately. For instance, an external display part and an external touch panel may be connected to the ophthalmic laser treatment apparatuses 1 and 2. In this case, upon receiving an operation command output from the external touch panel, the control unit 24 can make the same operations as in the first and second embodiments. It is further of course possible to use an operation part including various operation buttons instead of the touch panel 12. The operation part may be provided in the splitting unit 30 or the deflecting unit 130.

In the first embodiment, when either one of “SINGLE” and “MULTI” is selected by the operator, the number of laser beams to be irradiated from the tip of the probe 40 is selected. However, the method of receiving selection of the number of laser beams may be changed appropriately. For instance, the ophthalmic laser treatment apparatus 1 may be arranged to allow an operator to select the number of beams itself of the laser light to be irradiated from the tip of the probe 40. The same applies to the second embodiment.

In the ophthalmic laser treatment apparatus 1 of the first embodiment, for the irradiation pattern of “MULTI”, the laser beams are delivered to three of the four fibers of the probe 40. For the irradiation pattern of “SINGLE”, the laser beam is delivered to one of the four fibers, the one being different from the fibers selected for the “MULTI” pattern. However, the ophthalmic laser treatment apparatus 1 may be arranged to deliver the laser beam or beams to the same fiber or fibers for both the “MULTI” pattern and the “SINGLE” pattern.

As the method of changing the irradiation pattern by use of the splitting unit of the laser beam, a method of changing between open and block of the optical path of the split laser beams by a shutter or the like is also conceivable. In this case, more electric power will be wasted as the number of optical paths to be blocked is larger. However, it is unnecessary to adjust the output of the laser source 20.

The ophthalmic laser treatment apparatus 2 of the second embodiment is arranged to drive the galvano mirror 132 according to the gauge of the probe 40 to enable the control suitable for the number of fibers of the probe 40. However, the ophthalmic laser treatment apparatus 2 may be arranged to directly obtain the information on the number of fibers of the probe 40 instead of or together with the gauge information of the probe 40, and control driving of the galvano mirror 132 according to the number of fibers. In this case, for example, the ophthalmic laser treatment apparatus 2 may be arranged to obtain the information on the number of fibers by operation of the touch panel 12 by an operator. The control part 24 may also drive the galvano mirror 132 according to the number of fibers in the processings in S127 to S129 in FIG. 12. In this case, the ophthalmic laser treatment apparatus 2 can also make appropriate control suitable for the number of fibers of the probe 40. Furthermore, the ophthalmic laser treatment apparatus 2 of the second embodiment may also determine the diameter of each of the plurality of fibers based on the gauge information and drive the galvano mirror 132 according to the diameter of each fiber. The ophthalmic laser treatment apparatus 2 may be configured to obtain the gauge information of the probe 40 connected to the probe connector 33 from the shape of the rear end of the probe 40 and others.

The ophthalmic laser treatment apparatus 2 of the second embodiment drives the galvano mirror 132 to deflect (scan) the laser beam. As an alternative, the ophthalmic laser treatment apparatus 2 may deflect the laser beam by use of any structure other than the galvano mirror 132. For instance, any other structure (e.g., a polygon mirror) for scanning light may be used. Instead of the structure of scanning light, a structure of deflecting light by ultrasonic wave may also be adopted. The ophthalmic laser treatment apparatus 2 of the second embodiment, configured to scan the laser beam in one direction (one dimensionally), can deflect the laser beam by simple structure and control. As an alternative, the laser beam can also be scanned two-dimensionally. In this case, the ophthalmic laser treatment apparatus 2 can easily further increase the variations of the irradiation patterns.

The probe connector 33 of the second embodiment enables connection with the probe 40 so that the plurality of fibers of the probe 40 are positioned in straight line. Accordingly, the ophthalmic laser treatment apparatus 2 can increase the variations of the irradiation patterns by simple structure and processing that scans the laser beam one-dimensionally. However, the ophthalmic laser treatment apparatus 2 may not be arranged so that the plurality of fibers are arranged in straight line if the structure of scanning a laser beam two-dimensionally is included. The positioning part 75 for positioning the entrance ends of the fibers may also be changed in configuration. For instance, a polygonal connector may be adopted for the probe connector 33 to position the entrance end with respect to the probe connector 33.

In the probe 40 of the second embodiment, the arrangement of entrance ends and the arrangement of emission ends of the plurality of fibers are different from each other. Accordingly, the probe 40 of the second embodiment can simplify the structure and control to deflect the laser beam and also irradiate the laser beam from the tip in various patterns. As an alternative, the arrangement of entrance ends and the arrangement of emission ends of the plurality of fibers may be the same as each other. The probe 40 of the second embodiment is inserted in the patient's eye 5 in use. However, the techniques shown in the first and second embodiments are also applicable to a probe not inserted in the patient's eye 5 (e.g., a probe to be used with a tip being placed in contact with a cornea).

What is claimed is:

1. An ophthalmic laser treatment apparatus for irradiating a laser beam to a patient's eye from a tip of a probe, including:
a probe connector to which rear ends of a plurality of fibers of the probe are connected;
a splitting unit for splitting a laser beam generated by a laser source into a plurality of laser beams; and
a control unit for controlling the ophthalmic laser treatment apparatus,
wherein the control unit is configured to:
receive a command to select number of laser beams to be irradiated from the tip of the probe;
drive the splitting unit according to the received command representing the number of laser beams to switch number of delivered beams corresponding to number of fibers to which the laser beam generated by the laser source will be delivered, of the plurality of fibers of the probe connected to the probe connector, and
increase output of the laser source when the number of delivered beams is switched to an increased number and decrease the output of the laser source when the number of delivered beams is switched to a decreased number.

2. The ophthalmic laser treatment apparatus according to claim 1, wherein the control unit keeps an irradiation time, corresponding to a time from start to end in which the laser beam is generated by the laser source, constant before and after the number of delivered beams is switched.

3. The ophthalmic laser treatment apparatus according to claim 1, wherein the control unit sets the output of the laser source to N times when the number of delivered beams is switched to N times.

4. The ophthalmic laser treatment apparatus according to claim 1, wherein the probe connector includes a positioning part for positioning entrance ends of the laser beams in predetermined positions, the entrance ends being located one each at the rear ends of the fibers.

5. An ophthalmic laser treatment apparatus for irradiating a laser beam to a patient's eye from a tip of a probe, including:
a probe connector to which rear ends of a plurality of fibers of the probe are connected;
a deflecting unit for deflecting a laser beam generated by a laser source; and
a control unit for controlling the ophthalmic laser treatment apparatus,
wherein the control unit is configure to:
receive a command to select an irradiation pattern of the laser beam to be irradiated from the tip of the probe; and
control driving of the deflecting unit according to the received selection command to switch the fiber to which the laser beam will be delivered, of the plurality of fibers of the probe connected to the probe connector.

6. The ophthalmic laser treatment apparatus according to claim 5, wherein the probe connector is configured to connect the probe so that entrance ends of the laser beam located one each at the rear ends of the plurality of fibers are arranged in straight line.

7. The ophthalmic laser treatment apparatus according to claim 5, wherein the probe connector includes a positioning part for positioning entrance ends of the laser beam in predetermined positions, the entrance ends being located one each at the rear ends of the fibers.

8. The ophthalmic laser treatment apparatus according to claim 5, wherein the control part is configured to:
obtain information on number of the fibers of the probe connected to the probe connector; and
control driving of the deflecting unit according to the obtained information on the number of the fibers and the received selection command of the irradiation pattern.

9. The ophthalmic laser treatment apparatus according to claim 5, wherein the control unit is configured to:
obtain information on gauge of the probe connected to the probe connector; and
control driving of the deflecting unit according to the obtained gauge information and the received selection command of the irradiation pattern.

10. The ophthalmic laser treatment apparatus according to claim 5, wherein the control unit executes calibration to cause the laser beam to enter a target fiber by adjusting a driving operation of the deflecting unit when the laser beam deflected by the deflecting unit does not enter the target fiber of the plurality of fibers.
